SEQUENCE LISTING (11 sequences)

SEQ ID NO: 1 - protein, 101 aa, Human immunodeficiency virus type 1
MEPVDPRLEPWKHPGSQPKTACTTCYCKKCCFHCQVCFTKKALGISYGRKKRRQRRRAPEDSQTHQVSPPKQPAPQFRGDPTGPKESKKKVERETETHPVD

SEQ ID NO: 2 - DNA, 303 bp, Human immunodeficiency virus type 1
atggagcccgtggaccctcgcctggagccctggaagcacccgggcagccagcccaagaccgcctgcaccacatgttactgcaagaagtgctgcttccactgccaggtgtgcttcaccaagaaggccttgggcatcagctacggccgcaagaagcgccggcagcgccgccgggcccctgaggacagccagacccaccaggtgagccctcccaagcagcccgctccacagttccgcggcgaccctaccggtcccaaggagagcaagaagaaggtggagcgcgagaccgagacccatcccgtcgac

SEQ ID NO: 3 - protein, 17 aa, Human immunodeficiency virus type 1
PVDPRLEPWKHPGSQPK

SEQ ID NO: 4 - protein, 21 aa, Human immunodeficiency virus type 1
PCDWPLTPDPWVYSGSQPKVP

SEQ ID NO: 5 - protein, 27 aa, Simian immunodeficiency virus
PLREQENSLESSNERSSCILEADATTP

SEQ ID NO: 6 - protein, 11 aa, Human immunodeficiency virus type 1
SNERSSCELEV

SEQ ID NO: 7 - protein, 16 aa, Human immunodeficiency virus type 1
CTTCYCKKCCFHCQVC

SEQ ID NO: 8 - DNA, 50 bp, Human immunodeficiency virus type 1
ccagtagatcctagactagagccctggaagcatccaggaagtcagcctaa

SEQ ID NO: 9 - DNA, 63 bp, Mus musculus
ccatgtgactggcccctgaccccgcacccctgggtatactccgggggccagcccaaagtgccc

SEQ ID NO: 10 - DNA, 33 bp, Simian immunodeficiency virus
agcaacgagcggagttcctgcgagttagaggtg

SEQ ID NO: 11 - protein, 98 aa, Artificial
  Modified immunostimulatory Tat
MEPSNERSSCELEVPKTACTTCYCKKCCFHCQVCFTKKALGISYGRKKRRQRRRAPEDSQTHQVSPPKQPAPQFRGDPTGPKESKKKVERETETHPVD

(19) United States
(12) Patent Application Publication
(10) Pub. No.: US 2005/0244434 A1
(43) Pub. Date: Nov. 3, 2005

(54) Tat-based tolerogen compositions and methods of making and using same

(76) Inventor: David I. Cohen, Pelham, NY (US)

Correspondence Address: PRESTON GATES & ELLIS LLP, 1900 MAIN STREET, SUITE 600, IRVINE, CA, 92614-7319, US

(21) Appl. No.: 11/082,381

(22) Filed: Mar. 16, 2005

Related U.S. Application Data
(63) Continuation-in-part of application No. 10/456,865, filed on Jun. 6, 2003.
Application No. 10/456,865 is a division of application No. 09/636,057, filed on Aug. 10, 2000, now Pat. No. 6,667,151.
(60) Provisional application No. 60/553,733, filed on Mar. 16, 2004.
(60) Provisional application No. 60/649,021, filed on Jan. 31, 2005.
(60) Provisional application No. 60/148,526, filed on Aug. 12, 1999.

(51) Int. Cl. C12Q 1/70; A61K 39/21; C12N 15/867

(52) U.S. Cl. 424/208.1; 435/456

(57) ABSTRACT

A Tat-based tolerogen composition comprising at least one immunogenic antigen coupled to at least one human immunodeficiency virus (HIV) trans-activator of transcription (Tat) molecule wherein the immunogenic antigen can be a foreign or endogenous antigen or fragments thereof. Additionally methods of suppressing organ transplant rejection and methods of treating autoimmune diseases are provided.

### RELATED APPLICATIONS

The present application claims priority to U.S. Provisional Patent Application Ser. No. 60/553,733 filed Mar. 16, 2004, to U.S. Provisional Patent Application Ser. No. 60/649,021 filed Jan. 31, 2005 and to U.S. patent application Ser. No. 10/456,865 filed Jun. 6, 2003 which is a divisional of U.S. patent application Ser. No. 09/636,057 filed Aug. 8, 2000, now U.S. Pat. No. 6,667,151, the entire disclosures of which are incorporated herein by reference.

### FIELD OF THE INVENTION

The present invention relates to the field of immune modulation therapeutics and more specifically to tolerogen compositions useful in suppressing inappropriate immune responses using Tat-based antigen-specific tolerogen compositions. Specifically, the tolerogen compositions of the present invention are comprised of the human immunodeficiency virus trans-activator of transcription (Tat), or fragments thereof, conjugated to an immunogenic antigen. Additionally, methods of treating organ transplant rejection and autoimmune diseases with the tolerogen compositions of the present invention are provided.

### BACKGROUND OF THE INVENTION

Recently, significant advances have been made in understanding the human immunodeficiency disease (HIV) process. For many years, researchers have been unable to explain the seemingly immediate and profound destruction of the immune system following the initial HIV infection. Equally puzzling was a phenomenon seen in a few patients referred to as long term non-progessors (LTNP). In LTNP patients, viral loads are high and the virus can be isolated easily from the HIV target immune cells such as CD4+ T lymphocytes (referred to herein as T4 cells). However, unlike the majority of infected individuals who develop acquired immune deficiency syndrome (AIDS), the LTNP do not demonstrate significant reduction in their T4 cells and do not progress to AIDS.

One possible, non-binding, theory that may explain these two phenomena involves a non-structural protein (a protein encoded by the virus genome that is not actually part of the virus itself) called the trans-activator of transcription (Tat). Tat is a variable RNA binding peptide of 86 to 110 amino acids in length that is encoded on two separate exons of the HIV genome. Tat is highly conserved among all human lentiviruses and is essential for viral replication. When lentivirus Tat binds to the TAR (trans-activation responsive) RNA region, transcription (conversion of viral RNA to DNA then to messenger RNA) levels increase significantly. The Tat protein associated with lentivirus virulence will be referred to hereinafter as Tat, Recently, it has been demonstrated that Tat increases viral RNA transcription and it has been proposed that Tat may initiate apoptosis (programmed cell death) in T4 cells and macrophages (a key part of the body's immune surveillance system for HIV infection) and possibly stimulates the over production of alpha interferon (α-interferon is a well established immunosuppressive cytokine). These, and other properties of lentivirus Tat proteins, have led to considerable scientific interest in Tat's role in pathogenesis and to the present inventor's proposal that Tat may act as a powerful immunosuppressant in vivo.

A potential key to lentivirus Tat pathogenesis may involve in its ability to trigger apoptosis. Conventional Tat initiates apoptosis by stimulating the expression of Fas ligand (FasL, a monomeric polypeptide cell surface marker associated with apoptosis) on the T4 cell and macrophage surface. When FasL is cross linked by binding with Fas (the counter part to FasL which is also expressed on a wide variety of cell types), the apoptotic system is activated. Consequently, the death of these essential T4 cells and macrophages is accelerated, resulting in extreme immunosuppression. Thus, extracellular Tat's presence early in the course of HIV infection could reduce a patient's immune response, giving the virus an advantage over the host. Furthermore, the direct destruction of T4 cells and induction of α-interferon production could help explain the lack of a robust cellular immune response seen in AIDS patients, as well as accounting for the initial profound immunosuppression.

Further support for this concept is found in a surprising new observation made by the present inventor who has demonstrated the Tat protein isolated from long term non-progressors is different from C-Tat found in AIDS patents. The Tat protein found in LTNP is capable of trans-activating viral RNA, however, LTNP Tat (designated herein after as IS-Tat for immunostimulatory Tat) does not induce apoptosis in T4 cells or macrophages and is not immunosuppressive. Moreover, T4 cells infected ex vivo with HIV isolated from LTNP (such cell lines are designated Tat TcL) can result in the over expression of IS-Tat proteins, often to the virtual exclusion of other viral proteins, that are strongly growth promoting rather than pro-apoptotic. The tat genes cloned from these Tat TcLs reveal sequence variations in two tat regions, at the amino terminus and within the first part of the second exon. These surprising discoveries could help explain why HIV infected LTNP T4 cells do not die off at the staggering rate seen in HIV infected individuals that progress to AIDS.

Additionally, variants of Tat are found in lentiviruses which infect monkey species yet do not result in the development immunodeficiency and epidemic infection. These variant Tat proteins direct monocyte differentiation into DCs which stimulate CTL responses. These simian Tat variants, and other Tat variants that are not immunosuppressive, have been termed attenuated or immunostimulatory Tat (IS-Tat).

Based on the observations with long-term CD4+ Tat T cell lines (Tat TcL), clinical observations, and experiments in animals, attenuated Tat (more specifically IS-Tat or, alternatively, Tat proteins that have been chemically or physically altered) may act as an immune stimulant activating T4 cells inducing their proliferation. This principle may help to explain the stable T4 levels seen in LTNP. Moreover, attenuated Tat may be useful as an adjuvant when co-administered with other active vaccine components such as, but not limited to, vaccines for other viruses, bacteria, rickettsia and cancer cells.

Cancers and chronic infections are the most prominent examples of common human diseases that respond to immune-based treatments. Although infections were the first diseases to be controlled by immunization, a series of clinical trials in humans starting in the 1980s have established that an immune response, particularly of the cytotoxic T lymphocyte (CTL) arm of the immune system, could regress some human melanomas (Phan CQ, et al., Cancer regression and autoimmunity induced by cytotoxic T lymphocyte-associated antigen 4 blockade in patients with metastatic melanoma, Proc Natl Acad Sc. USA 100:8372-7, 2003) and renal cancers. These observations were broadened by the discovery that dendritic cells (DC), a specific class of antigen-presenting cells (APC), are particularly effective at initiating CTL activity against cancers and other diseases (Banchereau J et al., Dendritic cells as vectors for therapy, Cell 106:2714, 2001; Dalyot-Herman N et al., Reversal of CD8+ T cell ignorance and induction of anti-tumor immunity by peptide-pulsed APC, J Immunol 165:6731-7, 2000). Technologies that target and activate DC have yielded some early successes against human cervical pre-malignancies, caused by infection with Human Papilloma Virus (HPV) and human lung cancer. In contrast to chemotherapeutic drugs currently used against cancer, agents that provoke a CTL response against cancer potentially are accompanied by few side effects, owing to the great specificity of the immune response.

Efforts to develop immunotherapeutic drugs that treat cancer have been hampered by technical difficulties in targeting and activating DC to deliver and sustain the required entry signals to the CTL. Antigen targeting for the induction of a CTL response is a challenge insofar as natural processing requires that the antigen enter the cytoplasm of the cell in order to bind to the immune system's major histocompatibility complex (MHC) Class I antigen, a prerequisite to CTL activation because the ligand for activating the T cell receptor on CTL is a complex of antigen and MHC Class I. In almost all cases protein antigens, even when they are coupled with a DC co-activator, enter exclusively into the alternative MHC Class II antigen presentation pathway that excludes CTL stimulation. This can be overcome in part by peptide-based technologies, because peptides bind to MHC Class I that is already on the surface of the DC. However, this technology is non-specific and most peptides are poor DC activators which limits their efficacy as human treatments for cancer.

A limited group of biological proteins are known to stimulate a CTL response. Variants and derivatives of the Human Immunodeficiency Virus 1 (HIV-1) trans-activator of transcription (Tat) can stimulate this CTL response (Moy P et al., Tat-mediated protein delivery can facilitate MHC class I presentation of antigens, Mol Biotechnol 6:105-13, 1996; Fanales-Belasio E et al., Native HIV-1 Tat protein targets monocyte-derived dendritic cells and enhances their maturation, function, and antigen-specific T cell responses, J Immunol 168:197-206, 2002). Additional biologics that are currently known to directly trigger a CTL response are based on heat shock proteins (HSP) (Suzue K et al., Heat shock fusion proteins as vehicles for antigen delivery into the major histocompatibility complex class I presentation pathway, Immunol 94:13146-51, 1997; Stebbing J et al., Disease-associated dendritic cells respond to disease-specific antigens through the common heal shock protein receptor, Blood 102:1808-14, 2003), or on the outer coat protein of certain bacteria. Heat shock proteins have shown limited efficacy in the treatment of certain genital neoplasms related to HPV infection.

A large body of evidence implies that Tat is secreted from infected cells. Extracellular Tat is taken up by uninfected cells resulting in trans-activation of transcripts, a subset of which stimulate the cell (Frankel A D and Pabo C O, Cellular uptake of the Tat protein from Human Immunodeficiency Virus, Cell 55:1189-93, 1988) and a subset of which initiate programmed cell death. These observations demonstrate that Tat enters the cytoplasm of cells, where trans-activation is mediated, but they did not establish the key mechanism of entry via the receptor. The immediate immunosuppression that accompanies HIV infection has been attributed to Tat and has hindered the generation of successful HIV vaccines (Viscidi R P et al, Inhibition of antigen-induced lymphocyte proliferation by Tat protein from HIV-1, Science 146:1606-8, 1989; Cohen S S et al., Pronounced acute immunosuppression in vivo mediated by HIV-1 Tat challenge, Proc Natl Acad Sci USA 96:10842-47, 1999). Additionally, Tat suppression occurs at both the antibody level and at the T cell level and is antigen-specific. This distinguishes Tat-induced immunosuppression from other immunosuppressants currently used in human therapy, such as cyclosporine, that work exclusively on T cells.

Biological agents currently used to treat disease introduce foreign antigens (monoclonal antibodies, insulin, Factor VIII, organ transplants) into the body. An immune response against these antigens is undesirable because this immunity neutralizes, or in the case of organ transplants, rejects the foreign body in addition to causing collateral damage through allergic and autoimmune reactions. Recombinant proteins of human origin have been very successful in overcoming this problem and sustaining the efficacy of certain biological therapies such as insulin, Factor VIII, and monoclonal antibodies. However, even in these successes, undesired auto-antibodies can still accumulate over time that limit or terminate efficacy. Methods to ameliorate these undesirable immune responses have not yet been developed.

Current immunosuppression treatment regimens are primarily designed for organ transplantation where a highly immunogenic foreign body often with multiple foreign antigens (histocompatibility antigens) must be maintained for the life of the patient. Up till the present time, this involves non-specific suppression of the entire immune system with multiple agents. Physicians and researchers have devised therapeutic regimens where a balance between the side effects of the immunosuppressants and organ rejection can be reached. The most common side effects associated with common immunosuppressive cocktails, which can include corticosteroids, cyclosporine and azathioprine, include stunted growth, weight gain, bone marrow inhibition, anemia, low white blood cell count and kidney damage. The most serious side effects, however, are infection, particularly with viruses and tumor formation due to the non-specific nature of the immune suppression. Therefore there exists a need to improved antigen-specific immunosuppressive therapies.

Autoimmune diseases are a series of unwanted immune responses that selectively destroy tissues. Severe autoimmune diseases are chronic, debilitating, and life-threatening. In some cases, specific agents that provoke a particular type of autoimmune disease are becoming defined. Approximately 2.5 million individuals currently suffer from rheumatoid arthritis (RA) in the US alone. Severe RA accelerates death rates at least five-fold compared to the general population (Wolfe F et al., Predicting mortality in patients with RA, Arth Rheumatism 48:1530-42, 2003). Peptide fragments from collagen type II, an important structural component in undamaged joints, can provoke RA in animals and could be developed as tolerizing agents for use against human RA (Van den Steen P et al., Cleavage of denatured natural collagen type II by neutrophil gelatinase B reveals enzyme specificity, post-translational modifications in the substrate, and the formation of remnant epitopes in rheumatoid arthritis, FASEB J 16:379-89, 2002).

Therefore, there exists a medical need for compositions which can be used as vaccines to specifically stimulate desired immune responses, such as in infectious diseases or cancer, and other compositions that suppress inappropriate immune responses to certain therapeutic, diagnostic or prophylactic agents and in autoimmune diseases in an antigen-specific manner.

### SUMMARY OF THE INVENTION

For the purposes of clarification and to avoid any possible confusion, the HIV Tat as used in the tolerogen compositions of the present invention will be designated as either “Tat” for conventional immunosuppressive Tat protein and “Tat*” or “ox-Tat*” for Tat that is genetically or chemically derivatized so that it is stimulatory. Additional abbreviations for Tat used in this disclosure include sTat (soluble Tat) and C-Tat (conventional native immunosuppressive Tat from HIV).

In an embodiment of the present invention, a Tat-based tolerogen composition is provided in which at least one immunogenic antigen coupled to at least one human immunodeficiency virus (HIV) trans-activator of transcription (Tat) molecule. The immunogenic antigen can be a foreign antigen or an endogenous antigen and can additionally comprise a full length protein or a fragment thereof. Non-limiting examples of immunogenic antigens useful in the tolerogen composition of the present invention include insulin, monoclonal antibodies, carbohydrate antigens and Factor VIII.

In an embodiment of the present invention, the Tat protein and the immunogenic antigen are physically linked via a protein conjugation method to form the tolerogen composition. In another embodiment of the present invention, the Tat protein and the immunogenic antigen are linked through genetic engineering of their DNA to provide a recombinant protein to form the tolerogen composition.

In another embodiment of the present invention a method for suppressing organ transplant rejection is provided comprising administering at least one Tat-based tolerogen composition to a patient in need of an organ transplant. The Tat-based tolerogen composition can be administered by methods including perfusing the organ with the tolerogen composition, by implanting a device saturated with the tolerogen composition wherein the tolerogen composition is released into the transplanted organ or a combination of the two methods.

In yet another embodiment of the present invention, a method for reducing inflammation is provided comprising administering at least one Tat-based tolerogen composition to a patient in need thereof.

In another embodiment of the present invention, a method for treating autoimmune diseases is provided comprising administering at least one Tat-based tolerogen composition to a patient in need thereof wherein the autoimmune disease can be rheumatoid arthritis.

### BRIEF DESCRIPTION OF THE DRAWINGS

FIG. 1A-B depicts fluorescence activated cell sorter analysis of the results of Tat activation of monocytes according to the teachings of the present invention. Human peripheral blood monocytes were committed to differentiate into DCs through 5 days of culture in GM-CSF and IL-4. Committed DCs were cultured overnight either in medium alone (Control), LPS, or Tat, after which they were stained with an anti-CD86 antibody and analyzed by FACScan for CD86, a specific marker of DC activation, induction (A) or generalized activation (B, enlargement into box R2, shown for Tat-stimulated cells).

FIG. 2 depicts the enhancement of antigen-specific activation of CTLs by Tat*-antigen (Ag) complexes according to the teachings of the present invention. CTL activity was quantitated as the number of γ-interferon-secreting spot-forming colonies (SFC)/10⁶ plated cells using ELISPOT assays.

FIG. 3 depicts median fluorescence of monocytes, cultured for six days either with no stimulus (0), TNF-α, LPS, decreasing concentrations of C-Tat, or oxidized ox-C-Tat and stained with an anti-Fas ligand (FasL) monoclonal antibody (Mab) followed by a fluorescinated goat anti-mouse polyclonal antibody.

FIG. 4A-B depicts antibody titer to immunizing antigen administered with the tolerogen composition of the present invention (PT) or non-immunosuppressive ox-Tat* (Ag) at 2 weeks (A) and 6 weeks (B) after immunization.

FIG. 5 depicts fluorescence-activated cell sorter analysis of mouse peritoneal macrophages that were isolated either after in vivo thioglycolate stimulation (Stimulated+adjuvant) or without in vivo stimulation (resting). Mouse peritoneal macrophages were cultured for five days either in the absence of additional stimulation (C), with LPS or with Tat. Activation was determined as percent enlarged cells (M1 fraction).

FIG. 6 depicts stable suppression of antigen-stimulated T lymphocytes by Tat-Ag complexes two weeks after immunization with the tolerogen compositions of the present invention.

FIG. 7 depicts the antigen-specificity of Tat suppression according to the teachings of the present invention. Mice were immunized at day 0 and boosted at day 7 with an adjuvant emulsion containing either Tat (Ag+Tat), or with Ag Alone as control. At day 14, draining lymph node cells were harvested and stimulated with either specific or non-specific antigen and proliferation measured by ³H thymidine uptake (CPM) after four days of culture.

FIG. 8 depicts fluorescence-activated cell sorter analysis of human peripheral blood monocytes cultured for four days in control medium (Control), or medium containing Tat or LPS according to the teachings of the present invention. Harvested cells were doubly stained with a fluoresceinated anti-FasL Mab (αFasL-fitc) and with an anti-CD14 rhodamine labeled Mab. Cells were analyzed by FACScan for activation (forward scatter), CD14 expression (% macrophages, R2), and for induction of FasL (MFI). The T cell population is labeled R1.

FIG. 9A-B depicts the regulatory and immunosuppressive characteristics of Tat-activated macrophages according to the teachings of the present invention. (A) Human polymorphonuclear neutrophils (PBMC) from one individual (PBMCs #3) cultured for 5 days in either medium with tetanus antigen (Ag), antigen with the further addition of Tat (Ag+Tat) or Ag with Tat and recombinant sFas protein (Ag+Tat+sFas). The results are graphed as stimulation index (mean cpm stimulated culture/mean cpm medium control). (B) Proliferation of PBMCs cultured 6 days with either tetanus or Candida antigen alone (Ag), compared with cultures in which Tat (Ag+Tat), or Tat and the antagonistic anti-Fas antibody, ZB4, were added (Ag+Tat+αFas).

FIG. 10 depicts domain 1 of the Tat molecule, the signal transduction domain, amino acids 3-19.

FIG. 11 depicts domain 2 of the Tat molecule, the cysteine-rich ligand binding domain, amino acids 22-37.

FIG. 12 depicts domain 3 of the Tat molecule, the membrane translocation sequence, amino acids 47-57.

FIG. 13A-B schematically depicts the construction of vaccine and tolerogen cassettes according to the teachings of the present invention. Panel A: Domains of native Tat. Panel B: Varying antigen cassettes for the production of the vaccines or tolerogens of the present invention. The immunostimulatory or immunosuppressive functions of domain 1 (SH3 binding motif) will determine if the resultant protein is a vaccine (immunostimulant) or tolerogen (immunosuppressive).

FIG. 14 depicts tolerogen composition constructs according to the present invention specific for preventing immune responses to human or humanized monoclonal antibodies.

### DETAILED DESCRIPTION OF THE INVENTION

For the purposes of clarification and to avoid any possible confusion, the HIV Tat as used in the tolerogen compositions of the present invention will be designated as either “Tat” for conventional immunosuppressive Tat protein and “Tat*” or “ox-Tat*” for Tat that is genetically or chemically derivatized so that it is stimulatory. Additional abbreviations for Tat used in this disclosure include sTat (soluble Tat) and C-Tat (conventional native immunosuppressive Tat from HIV).

The present invention provides tolerogen compositions for induction of tolerance to foreign antigens. The present invention further provides methods for preventing and treating undesirable and inappropriate immune responses to foreign and endogenous antigens and autoimmune diseases with these tolerogen compositions. The tolerogen compositions of the present invention are based upon the Human Immunodeficiency Virus (HIV) trans-activator of transcription (Tat).

The tolerogen compositions of the present invention are constructed from Tat, or Tat fragments, conjugated to immunogenic antigens, or antigen fragments. The tolerogen compositions of the present invention can be constructed though a variety of means known to persons skilled in the art including, but not limited to, protein conjugation, avidin-biotin conjugation, specific cross-linking methods, creation of recombinant molecules and the like.

The present inventor has unexpectedly demonstrated that HIV-1 Tat mediates two independent activities, a receptor-mediated triggering event at the cellular surface and an intracellular trans-activation activity that controls antigen-presenting cell (APC) differentiation. The receptor-mediated triggering event mediated by Tat is specific to APC, committing them for activation and differentiation into highly immunosuppressive antigen presenting cell regulatory macrophages (AReg) or into dendritic cells (DC) that stimulate specific cytotoxic T lymphocytes.

Antigen-presenting cells, macrophages and dendritic cells are critical in the pathogenesis or response to a variety of diseases, disorders and undesired immune responses. Tat triggers monocytes to differentiate into antigen-presenting macrophages expressing molecules that specifically suppress the immune response to the presented antigen(s). Treatment for human diseases may introduce foreign antigens (biologicals, including but not limited to, monoclonal antibodies, insulin, and erythropoietin) or tissues (including organ transplants and stents) where an immune response to the foreign agent is not desired. In autoimmune diseases, certain of the body's own endogenous molecules are incorrectly recognized as foreign, resulting in extensive inflammation and tissue damage. In one example, degradation of collagen type II into immunogenic peptides can trigger rheumatoid arthritis (RA) in animals and has been associated with human rheumatoid arthritis. Considerable research has centered on reducing the immune response to these proteins. It is the non-binding theory of the present inventor that the antigen-specific macrophage-induced suppression attributed to Tat can be applied to the reduction of the undesired immune response to certain foreign and endogenous molecules using the tolerogen compositions of the present invention.

The tolerogen compositions of the present invention can be produced with antigens implicated in a variety of autoimmune diseases. Autoimmune diseases which are within the scope of treatment with the tolerogen compositions of the present invention include, rheumatoid arthritis, diabetes, systemic lupus erythematosis, multiple sclerosis, inflammatory bowel diseases, psoriasis, scleroderma and autoimmune thyroid diseases.

Additionally the tolerogen compositions of the resent invention have the potential to treat other immune mediator diseases such as inflammation including, but not limited to, ocular inflammation and cardiac inflammation.

Unlike the current immunosuppressive therapies, the tolerogen compositions of the present invention have the potential to suppress antigen-specific immune responses without immunocompromising the patient. This is particularly important when chronic immunosuppressive therapy is needed, such as following organ transplantation or during autoimmune disease. This antigen-specific immune suppression requires a high specific activity tolerogen, which can be produced according to the teachings of the present invention. In the absence of a thorough understanding of the structure of the Tat molecule and the mechanism of Tat suppression, it has not been possible to rationally design and test tolerogens that maintain the specificity and activity of Tat. The present invention provides immunosuppressive, antigen-specific tolerogen compositions, based on the Tat molecule, that have been designed and constructed using the recent findings on the Tat molecule by the present inventor. The tolerogen compositions of the present invention provide immune tolerance to a specific antigen exclusively while the remainder of the immune system remains intact and fully responsive.

The tolerogen compositions of the present invention can be stably produced as recombinant molecules or as direct conjugates of Tat protein, or fragments, to antigens. In one embodiment of the present invention, the DNA sequence of an antigen, to which tolerance or specific immunosuppression is desired, is inserted into a tolerogen expression cassette and the antigen-tolerogen construct is produced by growing the tolerogen expression cassette in the appropriate cell system such that a secreted protein composition is produced. An antigen which elicits an immune response in a mammal can be incorporated into the tolerogen composition of the present invention. Suitable antigens include, but are not limited to, endogenous molecules such as those that illicit inappropriate immune responses in autoimmune diseases and foreign antigens. Non-limiting examples of foreign antigens that commonly elicit immune responses that limit their therapeutic potential include, but are not limited to, monoclonal antibodies (Mabs), carbohydrates, insulin, blood clotting factors, growth factors and hormones, enzymes and other diagnostic, therapeutic or prophylactic proteins. Carbohydrate antigens suitable for use in the tolerogen composition of the present invention include, but are not limited to, sialic acids. Monoclonal antibodies suitable for use in the tolerogen compositions of the present invention include, but are not limited to, murine Mabs, human Mabs and humanized Mabs or Mabs produced from any mammal. Blood clotting factors suitable for use in the tolerogen compositions of the present invention include, but are not limited to, Factor VIII, Factor VII (rVIIa), Factor IX, Factor II, Factor VII, Factor IX, Factor X, von Willebrand Factor and Anti-inhibitor Coagulation Factor. Enzymes suitable for use in the tolerogen compositions of the present invention include, but are not limited to, asparaginase, collagenase, glutaminase, hyaluronidase, lysozyme, rhodanase, ribonuclease, β-lactamase, streptokinase, trypsin, uricase, urokinase, adenine deaminase, superoxide dismutase. Growth factors and hormones suitable for use in the tolerogen compositions of the present invention include, but are not limited to, human growth hormone, erythropoietin, granulocyte or macrophage stimulating factors, keratinocyte growth factor, interferons and interleukins.

In one embodiment of the present invention, tolerogen compositions are produced which induce antigen-specific tolerance to foreign molecules in stem cell transplants. In exemplary embodiment, tolerogen compositions comprising the Neu5Gc immunogenic non-human sialic acid (Martin M J et al., Human embryonic stem cells express an immunogenic nonhuman sialic acid. Nat Med 11:228-32, 2005) are made through physically linking the sialic acid to immunosuppressive Tat, or a fragment thereof, and administered to the patient prior to transplantation of cells bearing this antigen. In another embodiment of the present invention, if alloantigens are not fully defined, a class of immunosuppressive macrophages are generated ex vivo by co-culturing a patient's monocytes with tolerogen compositions and donor stem cells as a source of alloantigens. Seventy-two hours later (at a time when the macrophages first become suppressive) the transplant containing donor stem cells and the tolerogen composition is administered intravenously into the patient.

In another embodiment of the present invention, a tolerogen cassette includes the immunoglobulin variable heavy (VH) and/or variable light (VL) region genes from any Mab useful in the diagnosis, prophylaxis or treatment of disease. The tolerogen composition of the present invention is administered prior to and/or concurrently with the immunogenic biological agent in order to ensure an antigen-specific tolerized state in the patient.

In one embodiment of the present invention, Tat protein, or a fragment thereof, is chemically coupled to a desired tolerogenic antigen to produce a tolerogen composition. In a non-limiting example, these conjugates are simply linked using a widely known biotin-avidin system. Biotin, a vitamin, and avidin, a lectin, have a high affinity to one another such that proteins conjugated to biotin bind in a stable manner to proteins conjugated to avidin. Tat is biotinylated using methods well known to a person of ordinary skill in the art. Similarly, the antigen of interest is conjugated to avidin according to standardized methodology. When biotinylated Tat and avidin-Ag are combined under concentration and temperature conditions necessary for such a reaction, a Tat-Ag conjugate is formed. It is within the scope of the present invention to conjugate antigens and Tat by other methods known to those skilled in the art of protein chemistry.

The present inventor has surprisingly determined that Tat stimulates APCs, as opposed to T cells and other cell types, at picomolar concentrations that are physiologic for in vivo activity. In vitro, APCs are approximately 1000 times more sensitive to Tat than T4 lymphocytes. Due to this discovery, one barrier to the successful use of Tat as an immunotherapeutic, namely achieving concentrations attainable in vivo, has been overcome.

The immunosuppressive effects of Tat are mediated by macrophages. When stimulated by Tat, either by natural HIV-1 infection or by Tat uptake, macrophages induce the Fas ligand (FasL), which in turn induce the programmed cell death (apoptosis) of antigen-reacting, Fas-expressing helper T cells (Example 2). Tat enhances the viability of cultured murine macrophages as long as the macrophages were first activated in vivo compared with no prior activation and stimulated with relatively high concentrations of Tat. By comparison, LPS promotes the viability of murine macrophages independently from in vivo stimulation, and at the same concentration effective for human macrophages. The Tat tolerogen of the present invention produces a stable suppression of mouse lymphocyte proliferation and may also serve to suppress an antigen-specific immune response to a variety of foreign antigens.

The macrophages responsible for these responses have been identified as antigen presenting cell regulatory macrophages (ARegs). ARegs are also known as “alternatively activated” macrophages (Tzachenis D et al., Blockade of B7/CD28 in mixed lymphocyte reaction cultures results in the generation of alternatively activated macrophages, which suppress T-cell responses, Blood 99:1465-73, 2002). ARegs are stable macrophages expressing FasL and secreting the cytokines IL-10 and IL-6 (Novak N et al., Engagement of FcεRI on human monocytes induces the production of IL-10 and prevents their differentiation in dendritic cells, J Immunol 167:797-804, 2001; Zhang H et al., Induction of specific T cell tolerance by Fas ligand-expressing antigen-presenting cells, J Immunol 162:1423-30, 1999). AReg are stable and respond in an autocrine and paracrine manner to these two cytokines, as well as in a paracrine manner to IL-4. These cytokines accumulate and switch the immune response from TH1 (based on helper T lymphocytes) to TH2 (based on suppressive T lymphocytes). As these cytokines build up they overwhelm and suppress the immune response and explain why immune responses are normally self-limiting in an antigen-specific manner.

An unexpected observation is that 1,000 fold lower concentrations of Tat (500 pM) trigger this effect on the macrophages, as compared with the concentration required to initiate direct apoptosis of CD4+ T cells (approximately 500 nM). Therefore, at concentrations of Tat achievable as a systemically administered immunomodulator, the macrophage effect will preferentially occur over the T cell effect.

The Tat-mediated antigen-specific suppression of the present invention is mediated through trans- (intracellular) activation of a CD14+ FasL+ macrophage. Example 3 of the present invention demonstrates that, in human cells, Tat-activated macrophages are immunosuppressive ARegs. At low concentrations of Tat (50 nM), Tat-induced immunosuppression was not only fully reversed by the addition of soluble Fas, but under these conditions, Tat actually became slightly stimulatory (relative to antigen treatment alone). Antibodies to FasL reversed Tat immunosuppression of tetanus responses and enhanced the Candida response relative to Tat treatment alone. Suppression could be fully reversed (>95% of control) with the further addition of anti-IL-10 and anti-IL-6 antibodies to the cultures, both cytokines deriving from macrophages under these culture conditions. The non-binding theory of the present inventor is that a portion of Tat-induced immunosuppression is contributed by induction of FasL, although other Tat-induced factors also could participate in suppressing T cell proliferative responses, especially at higher concentrations of Tat.

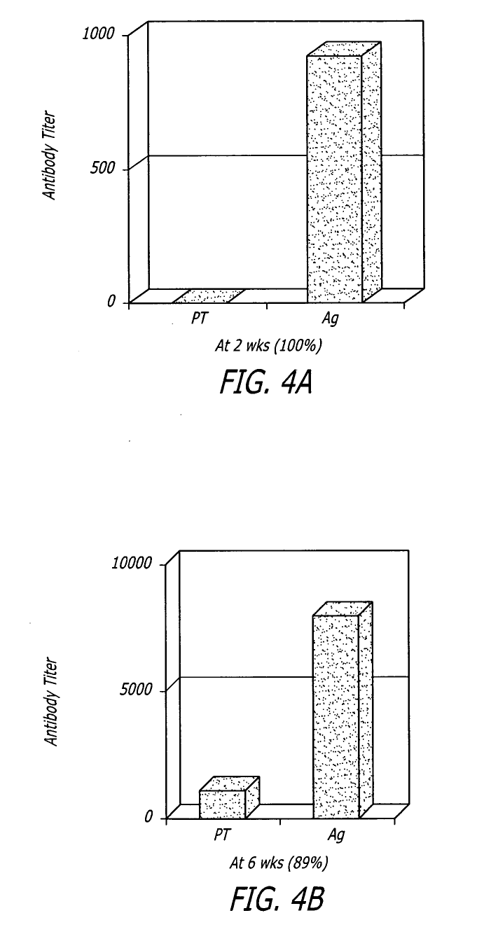

Additionally, a humoral immune response to HIV-1 p24 can be inhibited by including a p24-specific tolerogen made according to the teachings of the present invention (FIG. 4 and Example 2). Non-immunosuppressive ox-Tat* was included as a control. At two weeks after immunization, there was a 100% suppression of the anti-p24 response. This response was maintained at 6 weeks with an 89% suppression of the anti-p24 response.

When Tat-activated macrophages present more than one antigen, by uptake of soluble antigens, immune responses directed to these other antigens would be suppressed as well. This process will blunt T-cell dependent, cellular and humoral immune responses and can be harnessed to induce suppression of these responses in an antigen-specific manner by the administration of Tat-tolerogenic antigen complexes.

Previous obstacles to the use of Tat as an immunotherapeutic agent include the reported instability of the molecule and the disparity between Tat's activities in vitro and in vivo. The unexpected discovery of an activity and a preferred means to deliver mulitmerized Tat (to the APC) provides unique opportunities for drug development based upon increased specific activity. Although Tat is known to stably polymerize in vitro and in vivo, only the Tat monomer intracytoplasmically trans-activates gene expression (Tosi G et al., Highly stable oligomerization forms of HIV-1 Tat detected by monoclonal antibodies and requirement of monomeric forms for the transactivating function on the HIV-1 LTR, Eur J Immunol 30:1120-6, 2000).

Tat contains three distinct regions of interest (Kuppuswamy M et al., Multiple function domains of Tat, the trans-activator of HIV-1, defined by mutational analysis, Nucleic Acids Res 17:3551-61, 1989). The first region of interest is the transduction domain at the amino terminus of Tat (amino acids 3-19). A second region of interest is a cysteine-rich ligand binding domain (amino acids 22-37, SEQ ID NO. 7) which contains seven conserved cysteines. A third region of interest is the membrane translocation domain (MTS) which encompasses amino acids 47-57. The complete amino acid sequence of HIV-1 Tat encoded by exons 1 and 2 of the Tat gene is depicted in SEQ ID NO. 1.

A proline rich stretch near the amino terminus (amino acids 3-19) of HIV-1 and HIV-2 Tat (SEQ ID NO. 3) within the transduction domain, has been described as a new SH3 binding domain having significant homology to the SH3-binding domain of the mouse hairless gene (hr) (SEQ ID NO. 4). Unexpectedly, mice expressing the hr gene mutation develop an AIDS-like syndrome characterized by poor CTL function, a shift in helper T lymphocytes from those regulating cell-mediated immunity (TH1) to those regulating antibody-mediated immunity (TH2) and increased susceptibility to chemical and ultraviolet light-induced skin cancers. Additionally, variants of Tat are found in lentiviruses that infect monkey species that do not develop immunodeficiency and that do not have epidemic infection. However, these variant Tat do not have the SH3 binding domain and instead substitute a different sequence, also set off by prolines at either end of the sequence, into the transduction domain. Therefore, this SH3 binding domain is central to the immunosuppressive activity of Tat. Genetic data indicates this SH3 binding domain regulates monocyte differentiation into ARegs. In Tat proteins which do not contain this SH3 domain or is mutated, monocyte differentiation is directed into DCs which stimulate CTL responses.

It is also known that Tat contains a membrane translocation domain (MTS). After gaining access to the endosome following receptor binding, the MTS permits Tat to freely traffic across the endosomal membrane into the cytoplasm, where it transactivates gene expression, including but not restricted to genes of HIV-1 (Schwarze S R et al., In vivo protein transduction: delivery of a biologically active protein into the mouse, Science 285:1569-72, 1999). The MTS has been wrongly assumed to facilitate Tat entrance into the cell, which it can only accomplish at high concentrations that have been impossible to attain in vivo.

In an embodiment of the present invention, genetic derivatives of Tat, generated through modulating the signal transduction motif defined by the SH3 binding domain, are predicted to drive differentiation predominantly to dendritic cells or immunosuppressive AReg. AReg are also critical contributors to invasion of gastric, pancreas, and ductal infiltrating breast tumors, as well as components of tolerance in organ transplantation. It is a non-binding hypothesis of the present inventor that it is necessary to maintain the two external prolines at positions 3 and 18 flanking the SH3 domain in order to facilitate the proper structure for SH3 binding. In addition, the transduction domain from a non-immunosuppressive human variant Tat, or the domain from the hr mutation, can replace amino acids 3-19 of Tat, although the hr sequence (SEQ ID NO 4) is predicted to increase suppression. In addition, the stimulatory simian form of Tat (SEQ ID NO. 5), or its human equivalent sequence (SEQ ID NO. 6), can be substituted at this domain. Additional chemical modifications, such as ox-Tat, can be used for stimulation of dendritic/CTL responses and synthetic chemical moieties (NICE, new immunomodulatory chemical entities) can be constructed to generate an equivalent response.

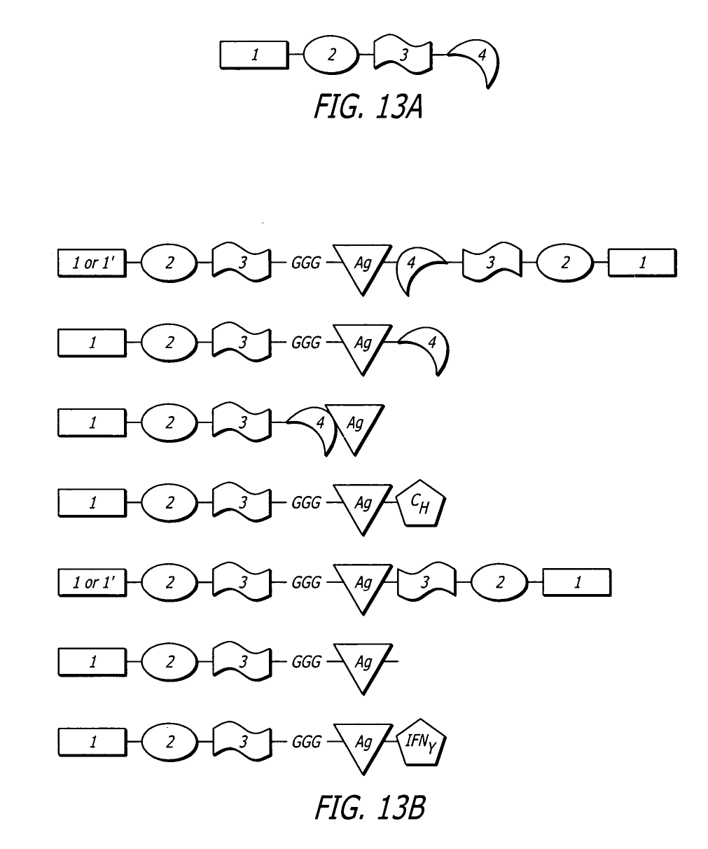

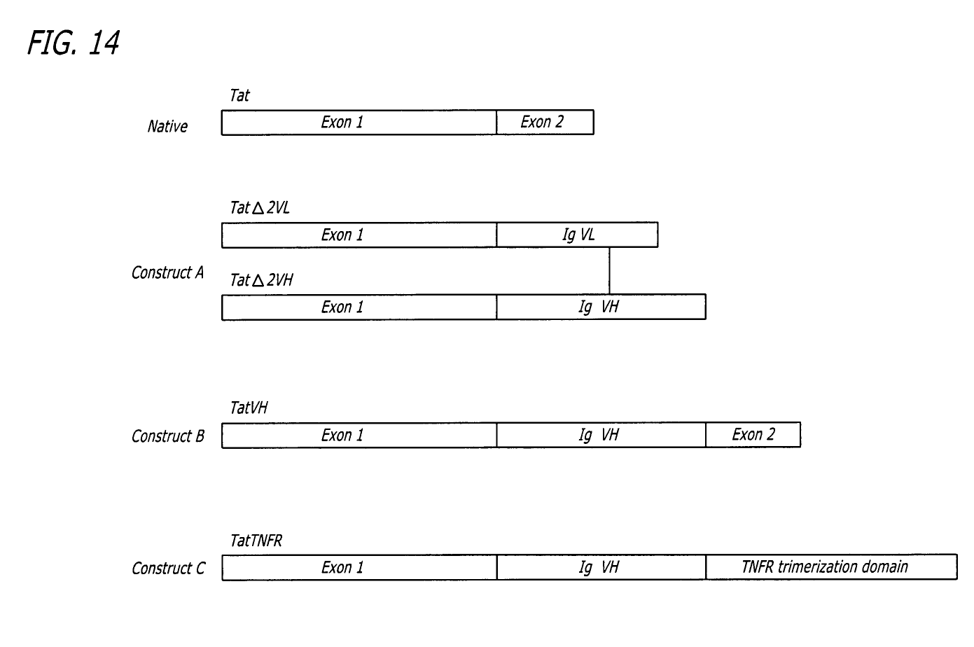

Variations of Tat for the purpose of inducing tolerance or immune suppression are proposed in where Tat is conjugated to antigen in one of several proposed configurations and further illustrated in FIGS. 13 and 14. The nature of the design allows the insertion of any specific antigen into a tolerogen cassette described here, in which tolerance will result to that antigen exclusively with an absence of effects on the remainder of the immune system. A particularly beneficial tolerogen construction would include the VH and/or VL regions from any Mab. In a non-limiting example, a Mab directed against a cancer growth antigen is used. In one embodiment of the present invention, constructs are generated where the tolerogenic antigen is sandwiched between two Tat molecules though linkage at the carboxyl terminus. One of the two Tat molecules is truncated at a point between amino acids 56 and 61. The resultant construct is a dimer with biological activity. In another embodiment of the present invention, constructs inserting the amino acid sequence for the tolerogenic antigen between amino acids 56 and 61. This “insertion” construct would then dimerize to provide a dimeric Tat with divalent antigen characteristics. The cassette could alternatively be trimerized through use of trimerization domains contained in DC stimulatory cytokines such as interferon-γ or TNF-α. Still another embodiment of the present invention is tolerogenic antigen linked to the carboxyl terminus of Tat.

Variations and derivitizations of Tat for the purpose of stimulating an immune response in a vaccine composition are proposed in which Tat is conjugated to antigen in one of several proposed configurations and further illustrated in FIG. 13. The nature of the design allows the insertion of any specific antigen into a vaccine cassette described here, in which a beneficial immune response will result to that antigen. FIG. 13A represents native immunosuppressive HIV Tat with four domains: (1) the transduction (SH3) domain (amino acids 3-19); (2) the cysteine-rich ligand binding domain (amino acids 22-37, SEQ ID NO. 7); (3) the membrane translocation sequence (amino acids 47-57) and (4) a tail portion encoded by the second exon (amino acids 73-101). In all potential conformations presented in FIG. 13B, domain 1 can be the native immunosuppressive Tat SH3 domain (1) or a modified or mutated immunostimulatory SH3 domain (1′). The conformational structures of FIG. 13B represent potential conformation by which recombinant compositions can be constructed to provide the desired functional activity. Other conformations are anticipated to be within the scope of the present invention. The target antigen (Ag) is included in both tolerogen and vaccine compositions. Other potential components of the compositions of the present invention include immunoglobulin chains, or fragments thereof (CH) or other effector molecules such as interferon-γ (IFNγ).

In one embodiment of the present invention, constructs for expression of tolerogen compositions are depicted in FIG. 14. Native Tat is comprised of two exons comprising 101 amino acids (SEQ ID NO. 1). Exon 1 encodes amino acids 1-72 and exon 2 encodes amino acids 73-101. Exon 1 encodes sequences that promote Tat stimulation of APC precursors (monocytes) and that modify DC differentiation. Tat can be stably dimerized (Construct A) by linking it to immunoglobulin (Ig) heavy (H) and light (L) chain variable (V) regions. These V regions being responsible for provoking the human anti-variable region antibody (HAVA) response which limits the safety and efficacy of monoclonal-antibody based therapeutics. Cysteine residues in the bridge (carboxyl end) region of the V regions promote the highly stable dimerization of the constructs. Construct B provides the same type of linked Tat-V constructs as Construct A using instead native Tat sequences for multimerization. This multimer is less stable than A but may dissociate more completely in the cell cytoplasm resulting in enhanced specific activity. Construct C uses the TNF receptor trimerization domain to yield a multimeric construct.

The nucleotide sequences representing the components of the constructs in FIG. 13 are constructed in expression vectors and expressed in cellular expression systems known to persons skilled in the art. One exemplary expression system is the baculovirus expression system including the transfer plasmids pPSC12 and pPSC10 and BaculoKIT™ expression system from Protein Sciences Corp. (Meriden, Conn.). It is anticipated that other expression systems, include eukaryotic and prokaryotic systems, are within the scope of the present invention.

In yet another embodiment of the present invention, tolerogen compositions which are determined to preferentially direct monocyte differentiation into ARegs will be evaluated for their ability to induce tolerance after being administered along with the desired immunogenic antigen. The tolerogen compositions of the present invention will be evaluated for their ability to induce tolerance in normal mice. Mice are injected with the tolerogen composition via a route including, but not limited to, intraperitoneal, subcutaneous, intradermal, oral, intranasal, cutaneous and intravenous administration. From four hours to one week after receiving a tolerizing agent, the mice are challenged with the corresponding immunogenic antigen alone. This test assay will be performed with an antigen which is known to induce an immune response in normal mice, such as a human protein. After an appropriate amount of time, ranging from 72 hours to 2 weeks, the mice are sacrificed and both T and B lymphocyte responses to the immunogenic antigen are determined using assays well known to those skilled in the art. The immune response in these mice will be validated by challenging the mice with an unrelated antigen which is known to induce an immune response (such as Candida) and with antigen that is not expected to induce an immune response (such as a normal mouse protein). Only if the mice react appropriately to these controls will the tolerogen composition be considered effective. In variations of the above experiment, additional mice will be administered multiple doses of tolerogen composition before challenging with corresponding immunogenic antigen. It is anticipated that repeated administration of the tolerogen composition will be necessary to induce and maintain tolerance to certain antigens and this schedule of dosing is optimized for each antigen.

An pharmaceutical method to influence the SH3 control of dendritic cells involves activating RNA interference (RNAi), which results in sequence-specific degradation of the targeted double strand RNA (Fire A, RNA-triggered gene splicing, Trends Genet 15:358-63, 1999; Zamore P D, RNA interference: listening to the sound of silence, Nat Struct Biol 8:746-50, 2001). Small interfering RNAs (siRNA) are RNA duplexes of 21-23 nucleotides which activate the RNAi pathway through their antisense strand and silence a gene through targeted degradation of its transcript. siRNAs are being widely developed as prophylactic and therapeutic agents to suppress selected RNA transcripts. Proposed targets include oncoproteins in cancer and infectious agents. The specificity and sensitivity of the target, an opening on the transcript free from secondary structure or complexed proteins that allows duplexed siRNA to form, and the actual delivery of the siRNA drug inside the cell are three critical factors governing the outcome of treatment. The sequence of the SH3 binding domain predisposing AReg/DC outcome is a potential RNAi target. Because the Tat's activity occurs at a balance point between stimulation (DC) and suppression (ARegs), small perturbations can be extremely efficacious.

An embodiment of the current invention is to create tolerogen compositions for organ transplantation using the genetic sequences discovered from analysis of Tat to control DC vs. AReg outcome. Duplexed siRNAs are easily constructed from the sense strand of Tat and Tat variants using methods standard to those skilled in the art (Elbashir S M et al., RNA Interference is mediated by 21- and 22-nucleotide RNAs. Genes Devel 15:188-200, 2001). One of the obstacles associated with the successful therapeutic use of siRNAs is the difficulty targeting the siRNA to the target cell. The signal transduction domain and the MTS of Tat are proposed as targeting agents for siRNA. The DNA sequences disclosed in Example 6 and in SEQ ID NOs. 8, 9 and 10 are exemplary Tat targeting sequences.

One of skill in the art will recognize that the efficacy, or toxicity, of the tolerogen compositions of the present invention, either alone or in combination with other pharmaceuticals, will influence the dose administered to a patient. Those of skill in the art may optimize dosage for maximum benefits with minimal toxicity in a patient without undue experimentation using any suitable method. Additionally, the tolerogen compositions of the present invention can be administered in vivo according to any of the methods known to those skilled in the art including, but not limited to, injection, inhalation, infusion and orally or any of the methods described in exemplary texts, such as “Remington's Pharmaceutical Sciences (8^{th} and 15^{th} Editions), the “Physicians' Desk Reference” and the “Merck Index.”

The tolerogen compositions can be formulated with any pharmaceutically acceptable excipient as determined to be appropriate by persons skilled in the art. Non-limiting examples of formulations considered with in the scope of the present invention include injectable solutions, lipid emulsions, depots and dry powders. Any suitable carrier can be used in the tolerogen composition, which will depend, in part, on the particular means or route of administration, as well as other practical considerations. The pharmaceutically acceptable carriers described herein, for example, vehicles, excipients, adjuvants or diluents, are well known to those who are skilled in the art and are readily available to the public. Accordingly, there are a wide variety of suitable formulations of the tolerogen composition of the present invention. The following formulations are exemplary and not intended to suggest that other formulations are not suitable.

Formulations that are injectable are among the preferred formulations. The requirements for effective pharmaceutical carriers for injectable compositions are well known to those of ordinary skill in the art (See Pharmaceutical and Pharmacy Practice, J. B. Lippincott Company, Philadelphia, Pa., Banker & Chalmers, Eds., pp. 238-50, 1982; ASHP Handbook on Injectable Drugs, Toissel, 4^{th} Ed., pp.622-30, 1986). Such injectable compositions can be administered intravenously or locally, i.e., at or near the site of a disease, or other condition in need of treatment.

Topical formulations are well known to those of skill in the art and are suitable in the context of the present invention. Such formulations are typically applied to skin or other body surfaces.

The tolerogen compositions of the present invention, alone or in combination with other suitable components can be made into aerosol formulations to be administered via inhalation. These aerosol formulations can be placed into pressurized acceptable propellants, such as dichlorodifluoromethane, propane, nitrogen and the like. The tolerogen compositions of the present invention can also be formulated for dry powder inhalers. They also may be formulated for non-pressured preparations, such as in a nebulizer or an atomizer. Such spray formulations are particularly suitable for spray application to mucosa.

Additionally, transplanted organs can be treated with the tolerogen composition of the present invention by implantation of a reservoir of tolerogen composition in close proximity to the transplanted organ such that the tolerogen composition is provided locally to the transplanted organ for an extended period of time such as days, weeks or months.

In addition to the above-described pharmaceutical compositions, the tolerogen compositions of the present invention can be formulated as inclusion complexes, such as cyclodextrin inclusion complexes, or in liposomes (including modified liposomes such as pegylated and/or targeted liposomes).

It is within the scope of the present invention to provide tolerogen compositions to a patient in need thereof through a plurality of routes of administrations using a plurality of formulations.

Additionally, the tolerogen compositions of the present invention can be administered to patients in need of antigen-specific immune suppression according to dosing schedules known to persons skilled in the art, such as physicians. The scope of the present invention is considered to include administration of the tolerogen compositions of the present invention either before, concurrent or after the patient has received a treatment with an immunogenic antigen. The tolerogen compositions may be administered in a single dose or as repeated doses.

### EXAMPLES

### Example 1

### Effects of Tat on the Dendritic Cell Lineage

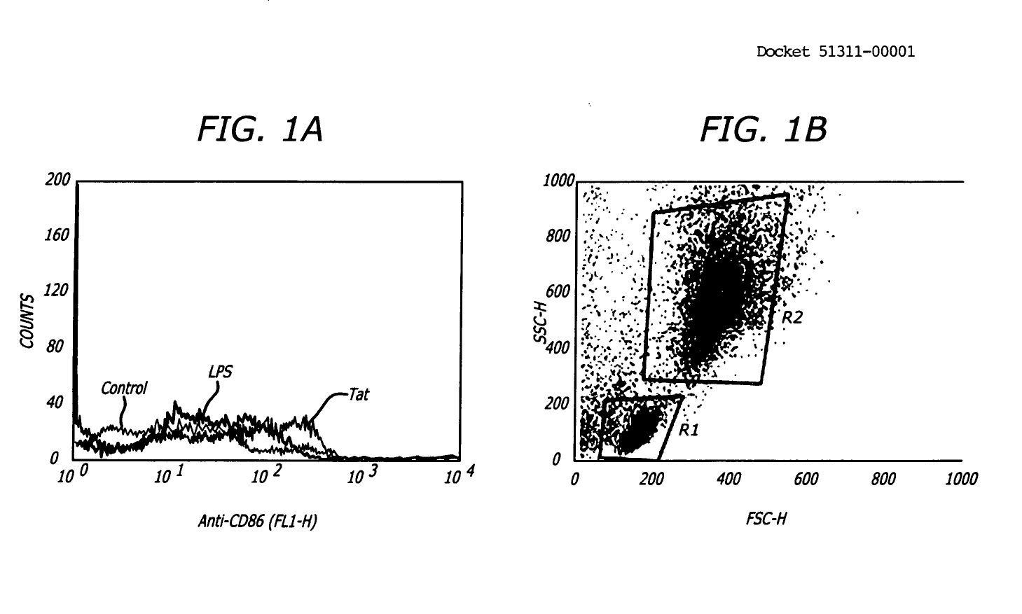

An additional embodiment of the present invention is that Tat induces monocytes committed to the dendritic cell (DC) lineage to enlarge into activated, CD86+ DC APCs (FIG. 1). Human monocytes enriched from PBMCs by Percoll density gradient separation and adherance to anti-CD14 coated magnetic beads (Dynabeads M450, Dynal Biotech) were committed to differentiate into DCs through five days of culture in GM-CSF (100 ng/mL) and IL4 (100 ng/mL). Committed DCs were cultured overnight either in medium alone (Control), LPS (100 ng/mL), or Tat (50nM), after which they were stained with an anti-CD86 antibody (BD Pharmingen) and analyzed by FACScan for CD86 induction (left panel) or generalized activation (right panel, enlargement into box R2, shown for Tat-stimulated cells). The MFIs for CD86 expression are 9 (Control), 30 (LPS), and 187 (Tat), CD86 being a specific determinant of DC activation.

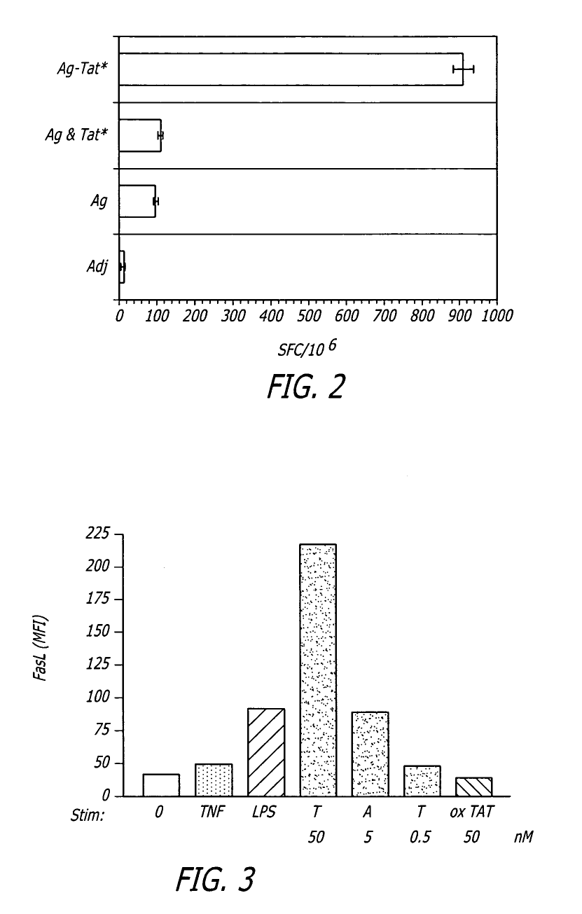

Derivitzed Tat reduces AReg differentiation and potently enhances antigen-specific activation of CTLs (FIG. 2). Tat is chemically derivatized by oxidation (Tat* or ox-Tat) so that it does not induce ARegs from monocyte APC precursors (FIG. 3). Ten micrograms of Tat/p24 Tat*-Ag conjugate (Ag-Tat*) was administered into the flanks of Balb/C mice in adjuvant on day 0 and day 7. Experimental groups were comparatively immunized in adjuvant with 5 μg of p24 in one flank and 5 μg derivatized Tat in the other flank (Ag & Tat*), or 10 μg of p24 in adjuvant (Ag). Control mice were given two injections of adjuvant. Four mice were treated in each group. At day 14, draining lymph node cells from each animal were harvested and re-stimulated overnight in cultures of irradiated Ap24 (H-2d cells stably transfected to express antigen p24) cells or control non-transfected cells. CTL activity was quantitated as the number of γ-interferon secreting spot forming colonies (SFC)/10⁶ plated cells using ELISPOT assays. The background with non-transfected re-stimulators, which was in all cases <10 SFC/10⁶, is subtracted from each point. The results are indicative of three similar experiments.

### Example 2

### Tat Activation of Macrophages and Suppression of the Immune Response

Recombinant Tat protein is prepared as previously described (Li, C. J. et al. (1995), “Induction of apoptosis in uninfected lymphocytes by HIV-1 Tat protein,” Science 268:429-31) under mildly denaturing conditions and was renatured in the presence of 0.1 mM DTT.

Tat activation of monocytes is dose-dependent and saturatable (FIG. 3). Human monocytes were cultured in increasing concentrations of recombinant Tat for six days at which time they were assayed for Fas ligand (FasL) induction as a measure of activation by using flow cytometry (FACScan, Becton Dickinson) to quantitate the intensity of staining (mean fluorescence index (MFI)) with an anti-Fas ligand monoclonal antibody (Nok 1, BD Pharmingen). Higher concentrations of Tat did not increase MFI (not shown), and T cells could not be activated with 50 nM Tat (not shown), the plateau stimulatory concentration for APCs.

Tat suppresses the antigen-specific humoral immune response to HIV-1 p24 (FIG. 4). At week 0, mice (4 in each group) were immunized with 5 μg recombinant p24 protein (Chiron, Emeryville, Calif.) and either 5 μg recombinant Tat protein (PT) or 5 μg recombinant ox-Tat* protein (Ag) mixed in 100 μL complete Freund's adjuvant and administered subcutaneously in the flank. Following immunization, sera were collected every other week for 10 weeks and assayed for a specific antibody response to p24 by commercially available ELISA (Abbott Laboratories, Abbott Park, Ill.). The p24 antibody titer at 2 weeks (FIG. 4A) was completely suppressed by the Tat protein (PT) compared with the ox-Tat* control (Ag). This response was maintained for at least 6 weeks. The antibody titers at 6 weeks are approximately ten times greater than at week 2 due to maturation of the immune response.

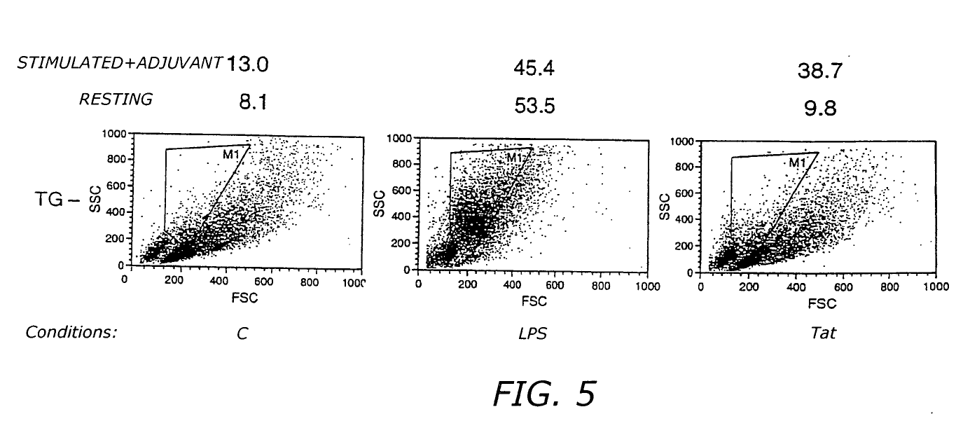

Tat enhances the viability of cultured murine macrophages as long as the macrophages were first activated in vivo compared with no prior activation and stimulated with relatively high concentrations of Tat (FIG. 5). APCs were isolated by peritoneal lavage from mice intraperitoneally injected four days earlier with either 2.9% thioglycolate (as adjuvant) or 0.85% saline solution (resting). Harvested washout cells were cultured at 10⁶ cells/mL for five days in medium alone (Control, C), lipopolysaccharide (LPS, 100 ng/mL), or Tat produced as recombinant protein in E. coli (Tat, 500 ng/mL). Activation was determined as % enlarged cells (M1 fraction).

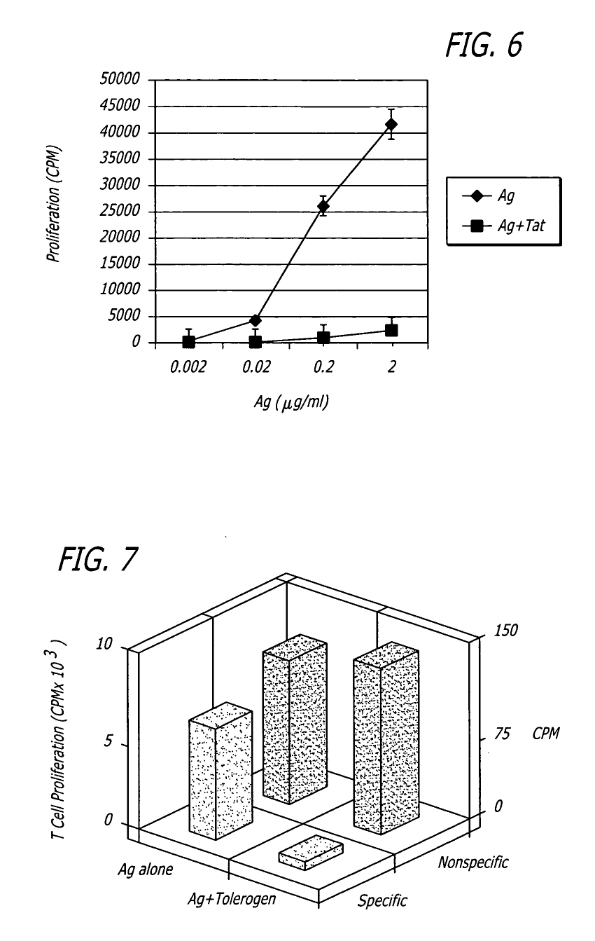

The Tat tolerogen of the present invention produces a stable suppression of mouse lymphocyte proliferation (FIG. 6). Mice were immunized in quadruplicate with a Freund's adjuvant emulsion containing either 5 μg Tat/p24 (recombinant HIV-1 gag protein p24) tolerogen (GRP 2) or with 5 μg avidin-p24 (GRP 1) as control. At two weeks residual draining lymph node cells were harvested, pooled within each group, and cultured at 10⁵ cells/microtiter well for four days in the presence of graded concentrations of recombinant p24 protein (p24, μg/mL). Proliferation was assayed as a determinant of recall T cell response by quantitating overnight ³H thymidine uptake (CPM) in a liquid scintillation counter. This response is maintained for up to six weeks.

In addition, the Tat tolerogen of the present invention generates an antigen-specific immune suppression (FIG. 7). Mice in quadruplicate were immunized at day 0 and boosted at day 7 with an adjuvant emulsion containing either 5 μg Tat/p24 tolerogen (Ag+Tol) or with 5 μg avidin-p24 (Ag Alone) as control. At day 14, draining lymph node cells were harvested and stimulated at 10⁵ cells/microtiter culture well either with added antigen (Specific, recombinant p24, 1 μg/mL) or with added anti-T cell receptor monoclonal antibody (NonSpecific, 2C11, 10 μg/mL). Tritiated thymidine uptake (CPM) was determined by liquid scintillation at day 4 of culture. The specific Ag+Tol response is suppressed 98% relative to Ag alone, and is not distinguishable from cells cultured in the absence of stimulants.

### Example 3

### Tat Suppression is Mediated by ARegs

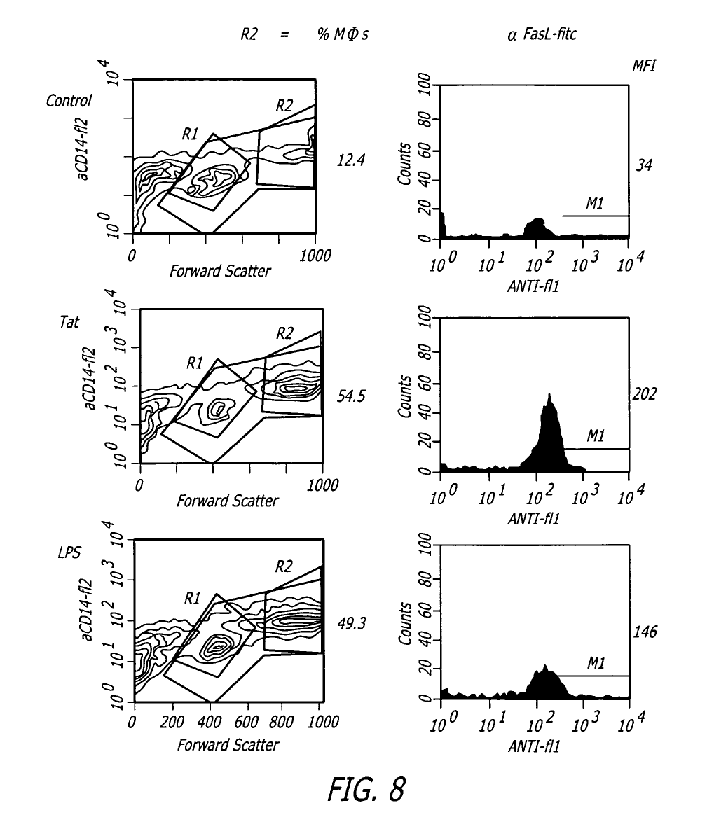

The Tat mediated antigen-specific suppression of the present invention is mediated through trans- (intracellular) activation of a CD14+ mFasL+ macrophage (FIG. 8). In mice, Tat tolerizes at the T cell level and is maintained for at least six weeks after the initial treatment under the conditions demonstrated in FIG. 6. A human peripheral blood mononuclear cell (PBMC) population enriched for monocytes by Percoll centrifugation was cultured for four days either in medium containing 5% fetal calf serum (FCS, Control), Tat (50 nM), or LPS (100 ng/mL). Harvested cells were doubly stained with a fluoresceinated (anti-fl1) anti-FasL monoclonal antibody (Mab), (αFasL-fitc, Nok 1, BD Pharmingen) and with an anti-CD14 rhodamine labeled Mab (αCD14fl2, BD Biosciences, CD14 being a determinant specific to macrophages (Mφ)). Cells were analyzed by FACScan (Becton Dickinson) for activation (Forward Scatter), CD14 expression (R2, percent Mφs), and for induction of FasL (MFI). The T cell population (R1) was CD14−and did not express FasL. Similar results were obtained from cells harvested after 2, 3, 5, or 6 days of culture as for PBMCs harvested at day four.

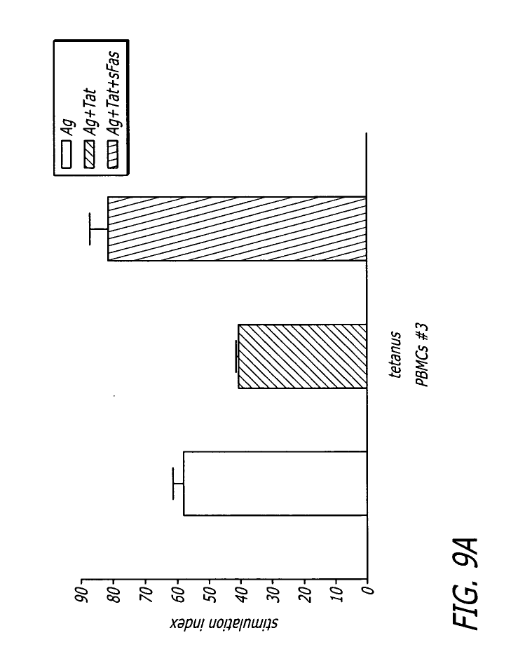

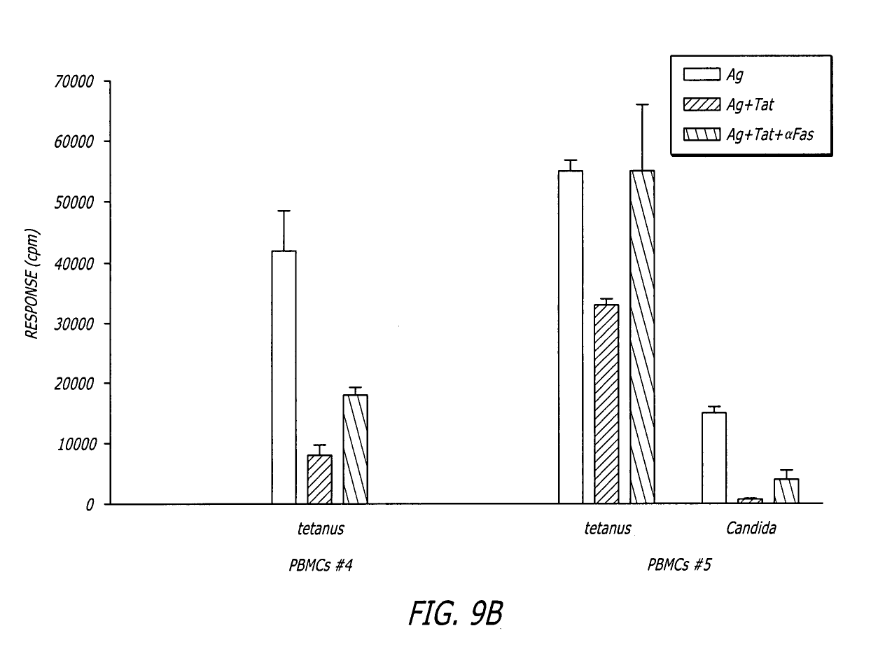

The present invention demonstrates that, in human cells, Tat-activated macrophages are regulatory and immunosuppressive APC macrophage regulators (ARegs) (FIG. 9). To define the pathway of Tat immunosuppression, through FasL induction on the macrophage, resulting in loss of helper T cell recall responses, T cell proliferation assays are used with recall antigens, tat and FasL antagonists. FIG. 9A: Human PBMCs from one individual were cultured in triplicate for 5 days in either medium (not shown), tetanus antigen (Ag, 0.3 Lf/mL), antigen with the further addition of 50 nM Tat (Ag+Tat) or Ag with 50 nM Tat and recombinant sFas protein (25 μg/mL) to block surface Fas L expressed on macrophages (Ag+Tat+sFas). Tritiated thymidine was added over the last 18 hours, and results are graphed as stimulation index (mean cpm stimulated culture/mean cpm medium control). Results are representative of three similar experiments. At low concentrations of Tat (50 nM), Tat-induced immunosuppression was not only fully reversed by the addition of soluble Fas, but under these conditions, Tat actually became stimulatory (141% relative to antigen treatment alone). FIG. 9B: Proliferation of PBMCs cultured 6 days with either tetanus or Candida antigen alone (Ag), compared with cultures in which Tat (Ag+Tat, 125 nM), or Tat (125 nM) and the antagonistic anti-Fas antibody, ZB4 (250 μg/mL, Upstate Biotechnology) also were added (Ag+Tat+aFas). Results are representative of three similar experiments.

### Example 4

### Sequence and Homology Features of the Tat Protein

The complete amino acid sequence of HIV-1 Tat encoded by exons 1 and 2 of the Tat gene is listed below:

| | |
|---|---|
| ATG GAG CCC GTG GAC CCT CGC CTG GAG CCC TGG AAG CAC CCG GGC AGC | SEQ ID NO. 1 |
| Met Glu Pro Val Asp Pro Arg Leu Glu Pro Trp Lys His Pro Gly Ser | SEQ ID NO. 2 |
| 1zzzzzzzzzzzzzzz5zzzzzzzzzzzzzzzzzzz10/30zzzzzzzzzzzzzzz15 | |
| CAG CCC AAG ACC GCC TGC ACC ACA TGT TACT GC AAG AAG TGC TGC TTC | |
| Gln Pro Lys Thr Ala Cys Thr Thr Cys Tyr Cys Lys Lys Cys Cys Phe | |
| zzzzzzzzzzzz20/60zzzzzzzzzzzzzzz25zzzzzzzzzzzzzzzzzz30/90 | |
| CAC TGC CAG GTG TGC TTC ACC AAG AAG GCC TTG GGC ATC AGC TAC GGC | |
| His Cys Gln Val Cys Phe Thr Lys Lys Ala Leu Gly Ile Ser Tyr Gly | |
| zzzzzzzz35zzzzzzzzzzzzzzzzzz40/120zzzzzzzzzzzzzz45 | |
| CGC AAG AAG CGC CGG CAG CGC CGC CGG FCC CCT GAG GAC AGC CAG ACC | |
| Arg Lys Lys Arg Arg Gln Arg Arg Arg Ala Pro Glu Asp Ser Gln Thr | |
| zzzz50/150zzzzzzzzzzzzzz55zzzzzzzzzzzzzzzzzz60/180 | |
| CAC CAG GTG AGC CCT CCC AAG CAG CCC GCT CCA CAG TTC CGC GGC GAC | |
| His Gln Val Ser Pro Pro Lys Gln Pro Ala Pro Gln Phe Arg Gly Asp | |
| 65zzzzzzzzzzzzzzzzzz70/210zzzzzzzzzzzzzz75zzzzzzzzzzzzzzzzzz80/240 | |
| CCT ACC GGT CCC AAG GAG AGC AAG AAG AAG GTG GAG CGC GAG ACC GAG | |
| Pro Thr Gly Pro Lys Glu Ser Lys Lys Lys Val Glu Arg Glu Thr Glu | |
| zzzzzzzzzzzzzzzz85zzzzzzzzzzzzzzzzzz90/270zzzzzzzzzzzzzz95 | |
| ACC CAT CCC GTC GAC | |
| Thr His Pro Val Asp | |
| zzzzzzzzzzzz100/300 | |

The Tat of the present invention has a proline (P) rich segment near the amino terminus (amino acids 3-19):

(SEQ ID NO. 3)

Pro Val Asp Pro Arg Leu Glu Pro Trp Lys His Pro
Gly Ser Gln Pro Lys

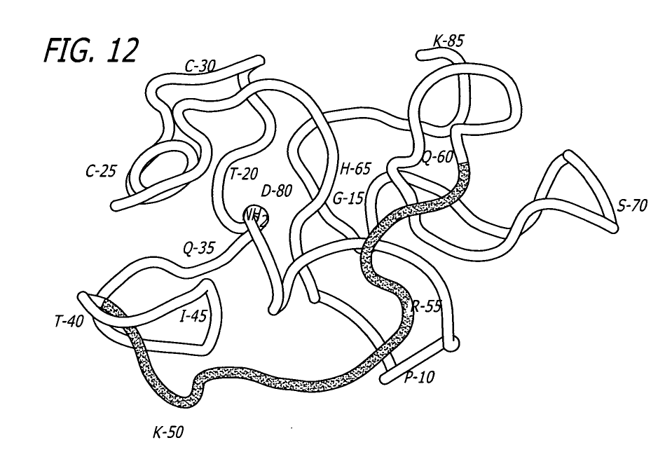

This highly conserved region of HIV-1 Tat is a canonical SH3 binding domain (FIG. 12).

The mouse hairless (hr) gene also has an SH3 binding motif of amino acids 176-196:

(SEQ ID NO. 4)

Pro Cys Asp Trp Pro Leu Thr Pro Asp Pro Trp Val
Tyr Ser Gly Ser Gln Pro Lys Val Pro

Homology exists between the human Tat SH3 binding domain (SEQ ID NO. 3) and the SH3 binding domain of the mouse hr gene (SEQ ID NO.4):

| | | |
|---|---|---|
| Human 3 | Pro Val Arg Pro Asn Leu Glu Pro Trp Lys His Pro | 14 |
| Mouse 180 | Pro Leu Thr Pro Asn --------Pro Trp Val Tyr Ser | 189 |
| Human 15 | Gly Ser Gln Pro | 18 |
| Mouse 190 | Gly Ser Gln Pro | 193 |

Variants of Tat found in simian lentiviruses that do not cause immunodeficiency do not have an SH3 binding domain but instead have the following proline-flanked sequence:

(SEQ ID NO. 5)

Pro Leu Arg Glu Gln Glu Asn Ser Leu Glu Ser Ser
Asn Glu Arg Ser Ser Cys Ile Leu Glu Ala Asp Ala
Thr Thr Pro

The human equivalent of the simian sequence above (SEQ ID NO.5) is:

(SEQ ID NO. 6)

Ser Asn Glu Arg Ser Ser Cys Glu Leu Glu Val

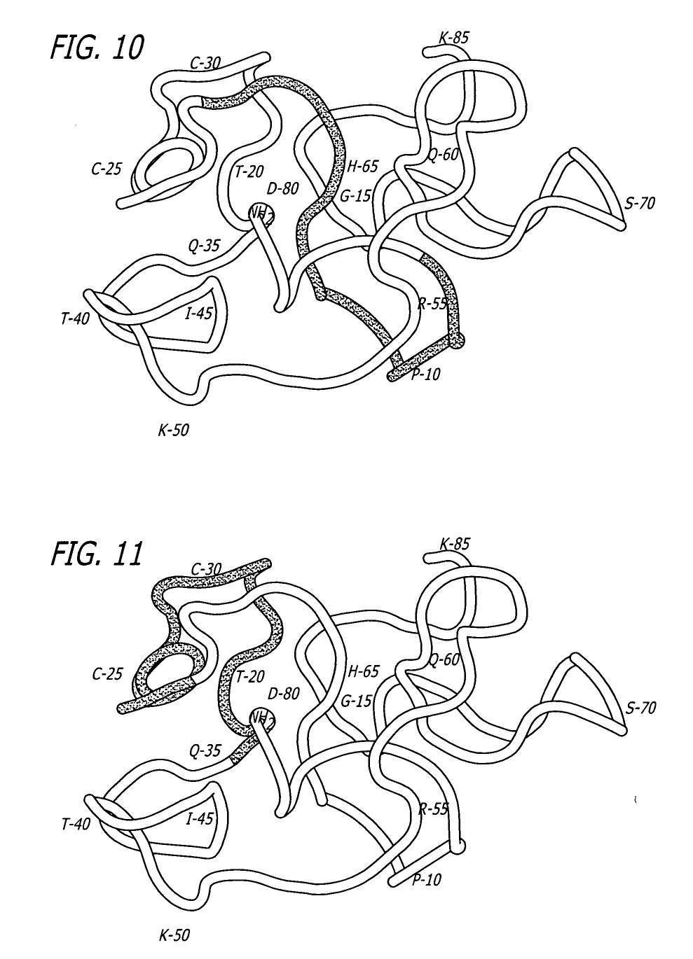

Another region of interest is a cysteine-rich proposed ligand binding domain (amino acids 22-37) which contains seven cysteines (FIG. 10).

(SEQ ID No. 7)

Cys Thr Thr Cys Tyr Cys Lys Lys Cys Cys Phe His
Cys Gln Val Cys

Additionally, it is known that Tat contains a membrane translocation domain (MTS) (FIG. 11).

### Example 5

### In Vitro Bioassay for Monocyte Differentiation

The in vitro ultra-sensitive monocyte Tat bioassay of the present invention is used to assess the immunosuppressant or immunostimulatory activity of the Tat proteins used in tolerogen compositions of the present invention. This assay utilizes fresh monocyte cells substantially purified from human peripheral blood using standard density gradient enrichment procedures or other cell isolation protocols known in the art. The substantially purified monocytes are washed and then cultured in RPMI-1640 supplemented with 10% FBS at 37° C.

The in vitro ultra-sensitive monocyte Tat bioassay is performed using a positive control (FasL, inducing compound) and a negative control (no active compound is added to the culture). Suitable positive controls include, but are not limited to, lipopolysaccharide (LPS) and or tissue necrosing factor (TNF-α) at a final concentration of 100 ng/mL and 50 ng/mL, respectively. Test samples (Tat preparations) are run at final concentrations from 50 pM to 50 nM and include Tat, ox-Tat, NICE and other Tat derivatives and mutants.

The test samples and controls are individually mixed with the substantially pure monocytes seeded at a density of 10⁶ cells/mL in round bottom tubes containing RPMI-1640 with 10% FBS (herein referred to collectively as assay cultures). The assay cultures are then incubated for a suitable period of time, preferably from five to six days, at 37° C., in a 5% CO₂ environment.

At the end of the incubation period, cells are removed from each assay culture and the presence of any induced FasL expression (for measurement of differentiation into ARegs) or CD86 expression (for differentiation in dendritic cells) is detected by staining with an anti-FasL or anti-CD86 antibodies and appropriate fluorescent detection agents. After the substantially pure macrophages have been stained, the fluorescence is detected using a fluorescence activated cell sorter (FACS) system. Control staining is performed using the fluorescent detection system alone and subtracted from the specific anti-FasL or anti-CD86 staining seen in the assay cultures. The greater the percentage of FasL positive cells in a given assay culture, the more immunosuppressant the test sample in the assay culture is. Conversely, if the assay culture contains a predominance of CD86 positive cells, the test sample is identified to be immunostimulatory. Negative controls should always remain non-reactive with the antibodies and the positive control should fall within predetermined ranges.

### Example 6

### siRNA Targeting Domains

Human Tat SH3 targeting domain:

(SEQ ID NO. 8)

ccagtagatc ctagactaga gccctggaag catccaggaa
gtcagcctaa

Mouse hairless SH3 targeting domain:

(SEQ ID NO. 9)

ccatgtgact ggcccctgac cccgcacccc tgggtatact
ccgggggcca gcccaaagtg ccc

Targeting domain from the human equivalent of the simian non-immunosuppressive Tat:

(SEQ ID NO. 10)

agcaacgagc ggagttcctg cgagctagag gtg

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A Tat-based tolerogen composition comprising at least one immunogenic antigen coupled to at least one human immunodeficiency virus (HIV) trans-activator of transcription (Tat) molecule.

2. The Tat-based tolerogen composition of claim 1 wherein said immunogenic antigen is a foreign antigen or an endogenous antigen.

3. The Tat-based tolerogen composition of claim 1 wherein said immunogenic antigen comprises a full length protein or a fragment thereof.

4. The Tat-based tolerogen composition of claim 1 wherein said immunogenic antigen is insulin or portions thereof.

5. The Tat-based tolerogen composition of claim 1 wherein said immunogenic antigen is a monoclonal antibody or portions thereof.

6. The Tat-based tolerogen composition of claim 1 wherein said immunogenic antigen is Factor VII or portions thereof.

7. The Tat-based tolerogen composition of claim 1 wherein said immunogenic antigen is a carbohydrate antigen.

8. The Tat-based tolerogen composition of claim 1 wherein said Tat protein and said immunogenic antigen are physically linked via a protein conjugation method.

9. The Tat-based tolerogen composition of claim 1 wherein said Tat protein and said immunogenic antigen are linked through genetic engineering of their DNA to provide a recombinant protein.

10. A method for suppressing organ transplant rejection comprising administering at least one Tat-based tolerogen composition to a patient in need of an organ transplant.

11. The method for suppressing organ transplant rejection according to claim 10 comprising perfusing said organ with said Tat-based tolerogen composition.

12. The method for suppressing organ transplant rejection according to claim 10 comprising implanting a device saturated with said Tat-based tolerogen composition wherein said Tat-based tolerogen composition is released into a transplanted organ.

13. The method for suppressing organ transplant rejection according to claim 11 additionally comprising implanting a device saturated with said Tat-based tolerogen composition wherein said Tat-based tolerogen composition is released into a transplanted organ.

14. A method for reducing inflammation comprising administering at least one Tat-based tolerogen composition to a patient in need thereof.

15. A method for treating autoimmune diseases comprising administering at least one Tat-based tolerogen composition to a patient in need thereof.

16. The method according to claim 15 wherein said autoimmune disease is rheumatoid arthritis.
